(19) United States
(12) Patent Application Publication
(10) Pub. No.: US 2019/0374123 A1
(43) Pub. Date: Dec. 12, 2019

(54) PREDICTING ATRIAL FIBRILLATION OR STROKE USING P-WAVE ANALYSIS

(71) Applicant: BRAEMAR MANUFACTURING, LLC, Eagan, MN (US)

(72) Inventors: Radouane Bouguerra, San Diego, CA (US); Wayne Derkac, Walloon Lake, MI (US); David Shanes, San Diego, CA (US)

(21) Appl. No.: 16/438,716

(22) Filed: Jun. 12, 2019

Related U.S. Application Data
(60) Provisional application No. 62/684,009, filed on Jun. 12, 2018.

(51) Int. Cl. A61B 5/046; A61B 5/00; A61B 5/0452; A61B 5/0472

(52) U.S. Cl. CPC A61B 5/046; A61B 5/7264; A61B 5/02405; A61B 5/0472; A61B 5/04525

(57) ABSTRACT

A system for predicting atrial fibrillation or stroke includes processing circuitry configured to receive ECG data from an ECG monitoring device, detect a first candidate P-wave represented in the ECG data, calculate a quality score of the first candidate P-wave by calculating a quality score of ECG data between the onset and the offset of the first candidate P-wave, detect a second candidate P-wave represented in the ECG data, calculate a quality score of the second candidate P-wave, compare the quality score of the first candidate P-wave with the quality score of the second candidate P-wave, store the candidate P-wave having the higher quality score, and calculate a characteristic of the stored P-wave, wherein the characteristic is at least one of a rate of rise of the stored P-wave, an area under the curve of the stored P-wave, and a duration of the stored P-wave.

### CROSS-REFERENCE TO RELATED APPLICATIONS

This application claims the benefit of priority from U.S. Provisional application entitled “Predicting Atrial Fibrillation Or Stroke Using P-Wave Analysis”, filed Jun. 12, 2018, Application Ser. No. 62/684,009.

### BACKGROUND

Various aspects of the functioning of the heart can be tracked by monitoring the heart's electrical activity. This electrical activity is commonly recorded as an electrocardiogram (ECG) using electrodes placed on a body surface. Anomalous electrical activity recorded in the ECG reports can be indicative of disease states or other physiological conditions, such as atrial fibrillation (AF). AF involves the loss of synchrony between the upper chambers of the heart (atria) and the lower chambers of the heart (ventricles). In complex AF, long-lived wave-like oscillations (wavelets) of depolarization travel along circular paths in the atria. This can lead to irregular ventricular contraction as well as blood stagnation. AF has been associated with cardiac disease as well as stroke.

Each ECG trace begins with a signal shape commonly referred to in the art as a “P-wave.” FIG. 2 shows a graphical example of an electrocardiogram signal 220, containing an example P-wave 222. P-waves mark the start of atrial contraction. More specifically, P-waves represent the electric potential (voltage) over time caused by depolarization of the atria during contraction. P-waves occur when a small body of specialized muscle tissue in the wall of the right atrium (the sinoatrial node) generate electric potentials (action potentials), causing a wave of contraction (depolarization) that spreads through the right and left atria along conduction pathways. Characterization of the P-wave can be used to understand the atrial structure and the state of AF. Conventional automated P-wave analysis tools typically rely on signal averaging, correlation-based template matching, and a number of empirical decision rules that are optimized for certain ECG databases, but which may cause signal degradation and loss of detail that is relevant to certain analyses.

### SUMMARY OF THE INVENTION

Analysis of P-waves may reveal certain latent atrial defects that may develop later into AF and/or stroke. Such latent atrial defects may be persistent, and unlike cardiac arrhythmia events (e.g., irregular/missed heartbeat, etc.), which may occur periodically and be more transient, signs of atrial defects may be imprinted on nearly every P-wave measured from a diseased atrium. Thus, a small number of P-waves, or even only a single P-wave, may be sufficient to detect such atrial defects. However, because some P-waves may not be representative of a patient's atrial function (e.g., P-waves generated during severe cardiac arrhythmia or obscured by motion artifacts), some processing or filtering of P waves may be necessary before being used to detect atrial defects. Lossy ECG processing and filtering techniques (e.g., averaging, correlation-based or empirical decision rules) may obscure certain characteristics in P-waves. For example, averaging techniques tend to smooth out high frequency components of the ECG trace. This may conceal some of the early signs of an atrial defect from the averaged ECG. Therefore, it is desirable to develop a system that selects representative high quality P-waves without losing important information (e.g., high frequency components) during processing.

Systems, methods, and devices for filtering and characterizing P-waves for the purpose of detecting atrial defects and predicting AF and/or stroke are presented herein. The systems, methods, and devices described herein include processing circuitry that identifies candidate P-waves, filters the candidate P-waves, and extracts one or more high quality P-waves, while preserving high frequency components of the candidate P-waves. The systems, methods, and devices also include circuitry which calculates P-wave characteristics based on the high quality P-waves. Such characteristics may be used for detecting atrial defects or determining an indication of the likelihood of the patient developing atrial fibrillation or stroke.

The present system may select high quality P-waves by comparing two candidate P-waves, storing the candidate P-wave having a higher quality, and discarding the candidate P-wave with a lower quality score. Discarding, as used in this application, includes deleting, reallocating, overwriting, ignoring, removing, excluding, and/or taking no action on, data. The quality score may be calculated in any number of suitable ways. In some embodiments, the quality score is based on the level of noise or distortion in the ECG signal. In some embodiments, the quality score is based on whether the P-wave was measured during a cardiac arrhythmia event. Alternatively, the system can have a quality threshold and can select P-waves that meet the threshold and discard P-waves that fail to meet that threshold. Discarding low quality samples can prevent unrepresentative or noisy P-waves from interfering with the analysis and detection of atrial defects. The system may also preserve the high frequency components of P-waves by identifying high quality P-waves without averaging or with less averaging than conventional approaches. For example, the present system can identify and record a set number (e.g., 1, 2, 3, 4, 5, 6, 7, 8, 9, 10, 15, 20, or any other suitable number) of high quality P-waves to produce an ECG report that includes a manageable number of waveforms such that a user could review each individually without averaging them together.

Additionally, the system calculates characteristics of the high quality P-waves for the purpose of assessing atrial structure and risk of AF and/or stroke. The calculated metrics can include a rate of rise of the P-wave, an area under the curve of the P-wave, a duration of the P-wave, and any other suitable measurement, or any combination thereof. From these measurements, an indication of the likelihood of atrial cardiomyopathy, impaired atrial conduction, other diseases of the atrial wall, or AF can be calculated. Since these conditions are closely associated with stroke, the risk of stroke may be determined based on the likelihood of AF.

In one aspect, an ECG monitoring system includes a pair of electrodes electrically coupled to an ECG sensor and configured to receive ECG data from a patient and a computer-readable memory. The ECG sensor is configured to receive the ECG data from the pair of electrodes; detect an onset and an offset of a first candidate P-wave represented in the ECG data; calculate a quality score of the first candidate P-wave by calculating a quality score of ECG data between the onset and the offset of the first candidate P-wave; detect an onset and an offset of a second candidate P-wave represented in the ECG data; calculate a quality score of the second candidate P-wave by calculating a quality score of ECG data between the onset and the offset of the second candidate P-wave; compare the quality score of the first candidate P-wave with the quality score of the second candidate P-wave; in response to determining, based on the comparison, that the first candidate P-wave has a lower quality score than the second candidate P-wave, discard the first candidate P-wave; store, in the computer-readable memory, an indication of the second candidate P-wave; and calculate a characteristic of the second candidate P-wave. The characteristic is at least one of a rate of rise of the stored P-wave, an area under the curve of the stored P-wave, and a duration of the stored P-wave.

In some implementations, calculating the quality score of the first candidate P-wave includes determining an average root mean square amplitude of noise between the onset and the offset of the first candidate P-wave. In certain implementations, calculating the quality score of the first candidate P-wave comprises determining whether the first candidate P-wave coincides with an arrhythmia event. In some implementations, the circuitry is configured to detect an onset of the first candidate P-wave using a Hilbert transform. In certain implementations, the circuitry is further configured to graphically present the calculated characteristic of the stored P-wave on a display. In some implementations, the circuitry is further configured to calculate an atrial conduction velocity based on the calculated characteristic of the stored P-wave. In certain implementations, the circuitry is further configured to calculate an indication of a likelihood of atrial fibrillation or stroke based on the characteristic of the stored P-wave. In some implementations, the circuitry is further configured to store in the computer-readable memory a plurality of P-waves.

In another aspect, a system for filtering P-waves includes processing circuitry configured to receive ECG data from an ECG monitoring device; detect an onset and an offset of a candidate P-wave represented in the ECG data; calculate a quality score of the candidate P-wave by calculating a quality score of ECG data between the onset and the offset of the candidate P-wave; compare the quality score of the candidate P-wave with a quality score threshold; if the quality score of the candidate P-wave is equal to or greater than the quality score threshold, store an indication of the candidate P-wave to computer-readable memory; and calculate a characteristic of the stored P-wave, wherein the characteristic is at least one of a rate of rise of the stored P-wave, an area under the curve of the stored P-wave, and a duration of the stored P-wave.

In some implementations, calculating the quality score of the candidate P-wave includes determining whether the P-wave coincides with an arrhythmia event, and the calculation of the quality score of the candidate P-wave is configured such that when the candidate P-wave does not coincide with an arrhythmia event, the calculated quality score of the candidate P-wave exceeds the quality score threshold. In certain implementations, the quality score of the candidate P-wave is an indicator of whether the candidate P-wave includes an ECG artifact, and wherein the quality score of the candidate P-wave is equal to or greater than the quality score threshold when the P-wave does not include an ECG artifact. In some implementations, the quality score threshold is determined based on at least one of: an error in the rate of rise of the stored P-wave, the area under the curve of the stored P-wave, or the duration of the stored P-wave. In certain implementations, the circuitry is further configured to graphically present the calculated characteristic of the stored P-wave in a display. In some implementations, the circuitry is further configured to calculate an indication of a likelihood of atrial fibrillation or stroke based on the characteristic of the stored P-wave. In certain implementations, the circuitry is further configured to store a plurality of P-waves in computer-readable memory.

In another aspect, a method for filtering P-waves includes receiving ECG data from an ECG monitoring device; detecting an onset and an offset of a first candidate P-wave represented in the ECG data; calculating a quality score of the first candidate P-wave by calculating a quality score of ECG data between the onset and the offset of the first candidate P-wave; comparing the quality score of the first candidate P-wave with a quality score threshold; if the quality score of the candidate P-wave is equal to or greater than the quality score threshold, storing the candidate P-wave; if the quality score of the candidate P-wave is less than the quality score threshold, discarding the candidate P-wave; and calculating a characteristic of the stored P-wave, wherein the characteristic is at least one of a rate of rise of the stored P-wave, an area under the curve of the stored P-wave, and a duration of the stored P-wave.

In some implementations, the method includes detecting an onset and an offset of a second candidate P-wave represented in the ECG data; calculating a quality score of the second candidate P-wave by calculating a quality score of ECG data between the onset and the offset of the second candidate P-wave; and setting the quality score threshold equal to the quality score of the second candidate P-wave. In certain implementations, the method also includes graphically presenting the calculated characteristic of the stored P-wave. In some implementations, the method includes estimating an atrial conduction velocity based on the calculated characteristic of the stored P-wave. In some implementations, the method includes estimating a likelihood of atrial fibrillation or stroke based on the characteristic of the stored P-wave.

### BRIEF DESCRIPTION OF THE DRAWINGS

FIG. 1 shows a schematic block diagram of an exemplary ECG monitoring environment in which some embodiments operate;

FIG. 2 shows a graphical example of an electrocardiogram signal;

FIG. 3 illustrates, according to an illustrative implementation, processing circuitry configured to operate on ECG and P-wave measurements;

FIGS. 4-6 illustrate, according to illustrative implementations, circuitry configured to compare the quality of P-wave measurements;

FIG. 7 shows, according to an illustrative implementation, a flow chart depicting a method for obtaining P-wave signals and comparing their quality to predict atrial fibrillation;

FIG. 8 shows, according to an illustrative implementation, a block diagram illustrating a P-wave detector in a cardiac monitoring apparatus;

FIG. 9 shows an example of an analytical signal for detection of a P-wave in state-space; and

FIG. 10 shows, according to an illustrative implementation, a flow chart of a method for determining whether P-waves are concurrent with stored cardiac arrhythmia events.

### DETAILED DESCRIPTION

Analysis of P-waves may reveal certain latent atrial defects that may develop later into AF and/or stroke. Such latent atrial defects may be persistent, and unlike cardiac arrhythmia events (e.g., irregular/missed heartbeat, etc.), which may occur periodically and be more transient, signs of atrial defects may be imprinted on nearly every P-wave measured from a diseased atrium. Thus, a small number of P-waves, or even only a single P-wave, may be sufficient to detect such atrial defects. However, because some P-waves may not be representative of a patient's atrial function (e.g., P-waves generated during severe cardiac arrhythmia or obscured by motion artifacts), some processing or filtering of P waves may be necessary before being used to detect atrial defects. Lossy ECG processing and filtering techniques (e.g., averaging, correlation-based or empirical decision rules) may obscure certain characteristics in P-waves. For example, averaging techniques tend to smooth out high frequency components of the ECG trace. This may conceal some of the early signs of an atrial defect from the averaged ECG. Therefore, it is desirable to develop a system that selects representative high quality P-waves without losing important information (e.g., high frequency components) during processing.

Systems, methods, and devices for filtering and characterizing P-waves for the purpose of detecting atrial defects and predicting AF and/or stroke are presented herein. The systems, methods, and devices described herein include processing circuitry that identifies candidate P-waves, filters the candidate P-waves, and extracts one or more high quality P-waves, while preserving high frequency components of the candidate P-waves. The systems, methods, and devices also include circuitry which calculates P-wave characteristics based on the high quality P-waves. Such characteristics may be used for detecting atrial defects or determining an indication of the likelihood of the patient developing atrial fibrillation or stroke.

The present system may select high quality P-waves by comparing two candidate P-waves, storing the candidate P-wave having a higher quality, and discarding the candidate P-wave with a lower quality score. Discarding, as used in this application, includes deleting, reallocating, overwriting, ignoring, removing, excluding, and/or taking no action on, data. The quality score may be calculated in any number of suitable ways. In some embodiments, the quality score is based on the level of noise or distortion in the ECG signal. In some embodiments, the quality score is based on whether the P-wave was measured during a cardiac arrhythmia event. Alternatively, the system can have a quality threshold and can select P-waves that meet the threshold and discard P-waves that fail to meet that threshold. Discarding low quality samples can prevent unrepresentative or noisy P-waves from interfering with the analysis and detection of atrial defects. The system may also preserve the high frequency components of P-waves by identifying high quality P-waves without averaging or with less averaging than conventional approaches. For example, the present system can identify and record a set number (e.g., 1, 2, 3, 4, 5, 6, 7, 8, 9, 10, 15, 20, or any other suitable number) of high quality P-waves to produce an ECG report that includes a manageable number of waveforms such that a user could review each individually without averaging them together.

Additionally, the system calculates characteristics of the high quality P-waves for the purpose of assessing atrial structure and risk of AF and/or stroke. The calculated metrics can include a rate of rise of the P-wave, an area under the curve of the P-wave, a duration of the P-wave, and any other suitable measurement, or any combination thereof. From these measurements, an indication of the likelihood of atrial cardiomyopathy, impaired atrial conduction, other diseases of the atrial wall, or AF can be calculated. Since these conditions are closely associated with stroke, the risk of stroke may be determined based on the likelihood of AF.

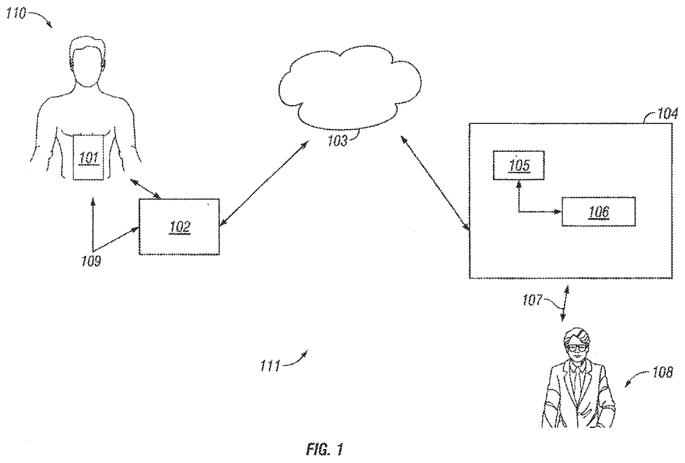

FIG. 1 shows a schematic block diagram of an exemplary ECG monitoring environment 111 in which some embodiments operate. The environment 111 includes a monitoring system 109, a communication network 103, a monitoring center 104, and a transmission path 107. The monitoring system 109 can be used by a user 108, such as a physician or other healthcare provider. The monitoring system 109 is worn by a patient 110 and includes a monitoring device 101 and a monitor processing device 102. The monitoring system 104 includes a monitoring (or display) station 105 and a processing system 106. The monitoring system 109 can communicate (via the devices 101 and 102) electrocardiogram (ECG) data, patient-initiated event data, and other data to the monitoring center 104. The monitoring system 109 includes, in some implementations, an implantable medical device (IMD), such as an implantable cardiac defibrillator and an associated transceiver or pacemaker and an associated transceiver. In some implementations, the monitoring device 101 is worn by the patient 110 or is patient-portable. In certain implementations, the monitor processing device 102 sends standard physiological data (received from the monitoring device 101) to the monitoring center 104 and can detect arrhythmia events, such as atrial fibrillation events and pause events. The monitoring system 109 may also record patient-initiated events, for example in response to the patient 110 pressing a button on the device 101, and transmit the patient-initiated event data to the monitoring center 104. In some implementations, the devices 101 and 102 are integrated into a single device. Moreover, the system 109 can be implemented using, for example, the CardioNet Mobile Cardiac Outpatient Telemetry (MCOT) device, which is commercially available and provided by CardioNet, Inc of Malvern, Pa.

The monitor processing device 102 can transmit physiological data, including data related to arrhythmia events, and patient-initiated event data through the communication network 103, which can be a local area network (LAN), a landline telephone network, a wireless network, a satellite communication network, or other suitable network to facilitate two-way communication with the monitoring center 104. Advantageously, the monitoring center 104 can be located in the same location (e.g., in the same room or building) as the monitoring system 109 or at some remote location.

The monitoring center 104 can include the monitoring (or display) station 105 and the processing system 106. In one implementation, a cardiovascular technician (CVT) can use the monitoring station 105 to evaluate physiological data received from the monitoring system 109, identifying and reporting, among other things, arrhythmia events (such as atrial fibrillation events). The CVT reports these assessments of the physiological data to the processing system 106, which also receives information related to the arrhythmia events identified by the monitoring system 109. As will be explained further below, the processing system 106 analyzes this arrhythmia event data (both the human-assessed data from the CVT and the data reported by the monitoring system 109) and determines whether to generate a report or pictographic presentation related to these events. In certain circumstances, the processing system 106 will send the report related to arrhythmia and/or patient-initiated events, for example, to the neurologist, physician, or other health care provider 108 via the transmission path 107, which may be part of the network 103.

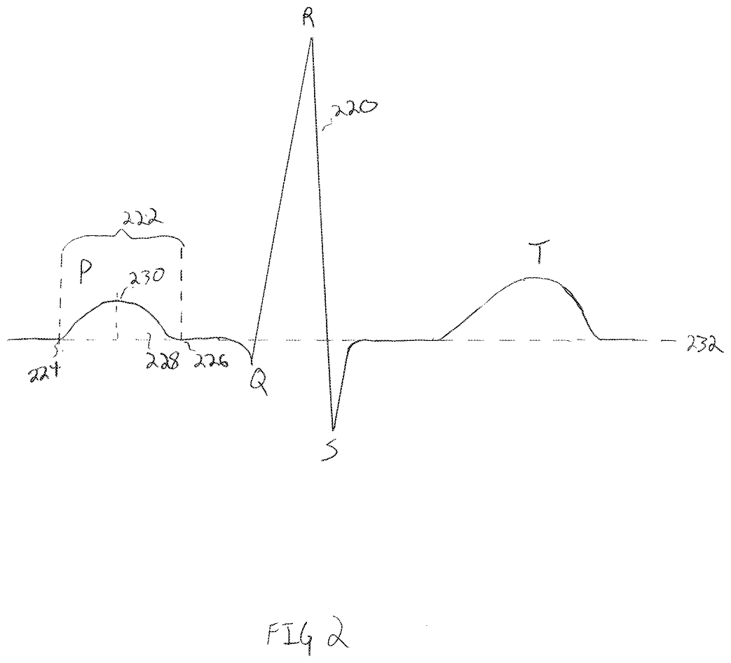

FIG. 2 shows a graphical example of an electrocardiogram signal 220. The ECG signal 220 is obtained by a monitoring system (e.g., monitoring system 106) and measured with respect to a reference line such as an isoelectric line 232. The ECG signal 220 contains a P-wave 222 that has an onset 224, an offset 226, an area under the curve 228, and a peak 230. As shown, the ECG signal 220 contains portions that are labeled with conventional lettering: P, Q, R, S, and T. The P-wave 222 is generated by atrial depolarization and the characteristics of the P-wave 222 can therefore lend insight into the structure and function of the atria, for example by revealing cardiomyopathy and/or impaired atrial conduction.

The onset 224 marks the initial rise of the P-wave 222, and the offset 226 marks the end of the down slope of the P-wave 222. The P-wave 222 can be defined as the portion of the ECG signal 220 between the onset 224 and the offset 226. Several characteristics of the P-wave 222 can be used for diagnostic purposes, including the duration, the area under the curve 228, the rise time, the rate of rise, or any other suitable characteristic. The duration of the P-wave 222 can be defined as the time between the onset 224 and the offset 226. The area under the curve 228 of the P-wave 222 can be defined as the area between the P-wave and the isoelectric line 232. The rise time of the P-wave 222 can be defined as the time from the onset 224 to the peak 230. The rate of rise of the P-wave 222 can be defined as the derivative of the P-wave 222 at a particular point (e.g., midway between the onset 224 and the peak 230) or the average derivative between the onset 224 and the peak 230, or in any other suitable manner. In some implementations, the peak 230, the onset 224, the offset 226, and/or any other fiducial point may also be used as a reference point for the alignment and comparison of different P-waves.

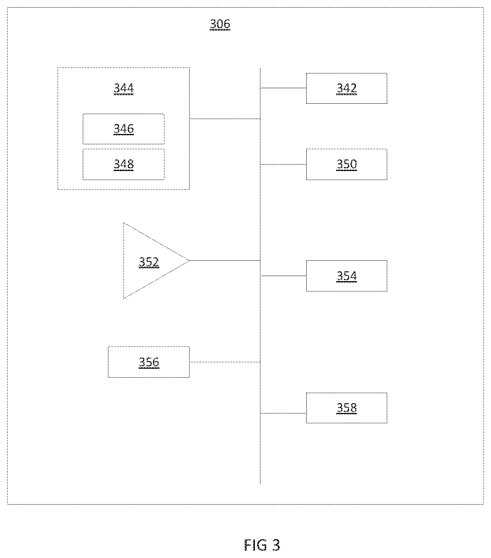

FIG. 3 illustrates, according to an illustrative implementation, the processing circuitry 306 configured to process ECG signals (e.g., ECG signal 220) and P-wave measurements. The processing circuitry 306 includes an ECG signal interface 342, a P-wave extractor 344, a P-wave quality score calculator 350, a P-wave filter 352, a memory 354 for storing P-waves, a P-wave characteristic calculator 356, an AF risk calculator 358, and a communication bus 340. The processing circuitry 306 can be included in the monitor processing device 102 of FIG. 1, the processing system 106 of FIG. 1, or in any other suitable processing system or device. The communication bus 340 connects the elements of the processing circuitry 306. The P-wave extractor 344 includes a P-wave onset detector 346 and a P-wave offset detector 348.

The ECG signal interface 342 is used to receive ECG data from a monitoring system (e.g., monitoring system 109) and communicate the ECG data as needed to the other elements of the processing circuitry 306, such as the P-wave extractor 344. The P-wave extractor 344 identifies P-waves within the received ECG signal. The identified P-waves may be excerpted from the ECG data or referred to by onset and offset timestamps or any other suitable means. In the illustrative implementation, the P-wave extractor 344 uses the P-wave onset detector 346 to determine the beginning of P-wave segments and the P-wave offset detector 348 to determine the end of P-wave segments. In some implementations, the P-wave extractor 344 uses a second biological signal, e.g. the Hilbert transform of the ECG signal, to form an analytic pair with the ECG signal. By analyzing the time varying phase angle of the analytic pair and defining reference lines, the P-wave extractor 344 can identify the onset and offset of a P-wave. In some implementations, the P-wave onset detector 346 defines an onset reference line and the P-wave offset detector 348 defines an offset reference line. Systems and methods for locating fiducial points in a physiological signal are described in U.S. Pat. No. 8,200,319, which is incorporated herein by reference in its entirety.

Once identified, data indicating the P-waves is communicated to the P-wave quality score calculator 350 that calculates a quality score to the P-wave. In some implementations, the calculated quality score is an integer, real number, scalar value, percentage, binary value, noise measurement (e.g. an RMS noise power), or any other suitable measure. The quality score can indicate the amount of noise and/or artifacts in the P-wave. In some implementations, P-waves containing measurement defects or interference artifacts are rejected. Systems and methods for determining the quality, or merit, of a biological signal are described in U.S. patent application Ser. No. 13/904,971, which is incorporated herein by reference in its entirety. In certain implementations, the quality score is an indication of whether the P-wave was measured during a cardiac arrhythmia event (e.g., atrial fibrillation, ventricular fibrillation, tachycardia, or a pause event). The absence of a cardiac arrhythmia event coinciding with the P-wave may be indicative of a more representative, and hence higher quality, P-wave. In certain implementations, the quality score is an indication of whether the P-wave coincided with an artifact (e.g., skeletal muscle contractions or respiration). In some implementations, the quality score is an indication of whether ECG leads are properly connected. The quality score may be binary (e.g., high quality and low quality). The noise estimate and/or quality score for a P-wave may be computed by any suitable means.

P-waves and their associated quality scores are communicated to the P-wave filter 352, which is configured to process P-wave signals based on their quality score. In some implementations, the filter 352 takes two P-waves as input and selects the higher quality P-wave. The lower quality P-wave may be discarded. As noted above, “discarding,” as used in this application, includes deleting, reallocating, overwriting, ignoring, removing, excluding, and/or taking no action on, data. In some implementations, P-waves are compared to a threshold quality score and P-waves with a quality score that exceeds the threshold are selected. In such implementations, the threshold may be predetermined based on an acceptable error in the measurement of the rate of rise of the stored P-wave, the area under the curve of the stored P-wave, or the duration of the stored P-wave. In certain implementations, the quality score of the candidate P-wave is an indicator of whether the candidate P-wave coincides with an arrhythmia event, and the quality score of the candidate P-wave meets the quality score threshold when the P-wave does not coincide with an arrhythmia event. In some implementations, the quality score of the candidate P-wave is a binary indicator of whether the candidate P-wave includes an ECG artifact, and the quality score of the candidate P-wave meets the quality score threshold when the P-wave does not include an ECG artifact. In some implementations, the filter 352 is configured as an ordered data structure that maintains a set or list of the highest quality P-waves identified. Identified P-waves are stored in the memory 354. In certain implementations, the memory 354 stores one or more sufficiently high quality P-waves that may be held in a data structure ordered by quality score. In some implementations, the memory 354 is used to store identified P-waves before they are compared.

Sufficiently high quality P-waves are communicated to the characteristic calculator 356, which calculates characteristics of the P-waves, such as the duration, the area under the curve (e.g., area under the curve 228), the rise time, a rate of rise, or any other suitable characteristic. Characteristics of the P-wave may also be calculated based on the P-wave as represented after the application of a waveform transform, a state-space transform, or any other suitable mathematical operation. The high quality P-waves, or the characteristics thereof, may be combined or averaged in any suitable manner. P-wave data is communicated to the AF risk calculator 358 and used to compute data relevant to AF. In some implementations, the data relevant to AF includes an atrial conduction velocity. In some implementations, the data relevant to AF includes a likelihood of AF. The AF risk calculator 358 may use a machine learning classifier, statistical regression, or any other suitable means to calculate AF likelihood and atrial conduction velocity. In some implementations, the calculated characteristics of the P-wave, AF likelihood, and atrial conduction velocity are graphically, or pictographically, presented to a user. A system that identifies and records only a set number of high quality P-waves can produce a report having a manageable number of representative waveforms (e.g., 1, 2, 3, 4, 5, 10, 20, >20, or any other suitable number) such that a user could review each. Although P-waves may be averaged, the systems and methods of the present disclosure allow for the use of un-averaged P-wave samples. For example by selecting a set of high quality P-waves, or a single high quality P-wave, averaging techniques may not be necessary. Using un-averaged P-wave data allows for a high resolution to be achieved because the high frequency information in the signal is not eliminated or distorted by the smoothing effects of averaging or other processing. The systems and methods of the present disclosure can also reduce or eliminate the effects of low quality samples distorting a composite P-wave, e.g., one obtained through averaging, or distorting a prediction based on a single P-wave.

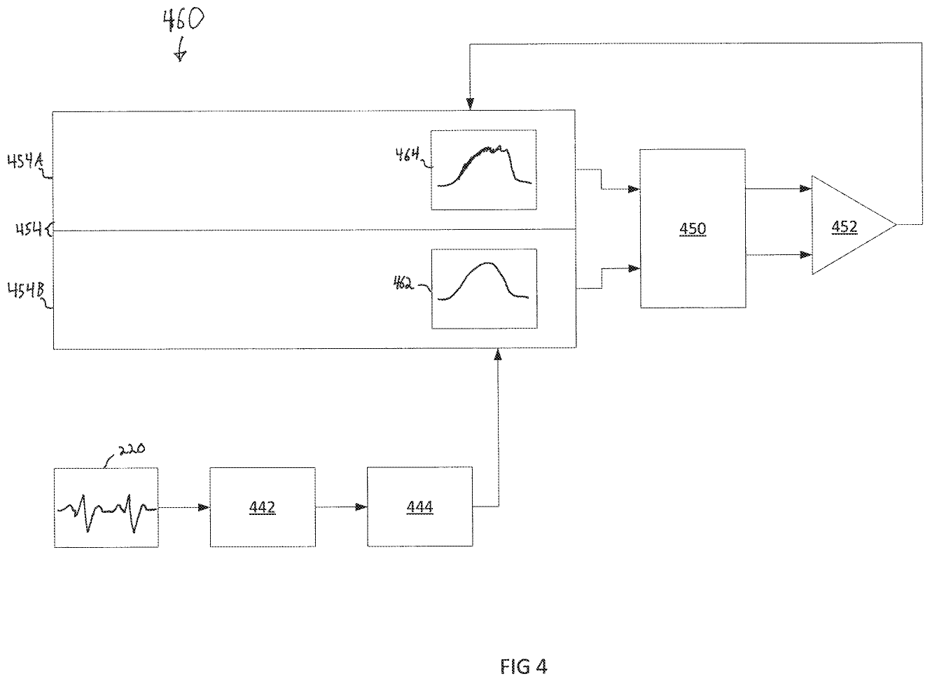

FIG. 4 illustrates, according to an illustrative implementation, circuitry 460 configured to compare the quality score of P-wave measurements. The circuitry 460 includes an ECG signal interface 442, a P-wave extractor 444, a memory 454, a P-wave quality score calculator 450 connected to the memory 454, and a P-wave filter 452. The memory 454 is segmented into a memory segment 454a, which stores P-wave 464, and a memory segment 454b, which stores P-wave 462. The ECG signal (e.g., ECG signal 220) is obtained from a patient by a monitoring system (e.g., monitoring system 109) and received by the ECG signal interface 442. The received ECG data is communicated to P-wave extractor 444, which identifies an onset and offset of a P-wave, extracts the P-wave 462 from the ECG data, and stores the P-wave 462 in the memory segment 454b. The P-wave 462 is the most recently obtained P-wave sample. The P-wave extractor 444 may extract every discernible P-wave in the ECG signal or a subset thereof.

The memory segment 454a and the memory segment 454b are connected to the P-wave quality score calculator 450, which receives extracted P-waves 462 and 464 as input and calculates a P-wave quality score for each. In some implementations, the quality score of P-waves whose quality score has already been calculated (P-wave 462 in this example) may pass through or bypass the P-wave quality score calculator 450 without duplicating any calculations. In some implementations, the quality score of P-waves is calculated on a relative basis. The calculation of P-wave quality scores may be performed in any suitable manner. The P-wave quality score calculator 450 outputs the quality scores for P-waves 462 and 464 to the P-wave filter 452. The P-wave filter 452 receives the P-waves and the quality scores of the P-waves and then outputs the P-wave having the higher quality score to memory segment 454a.

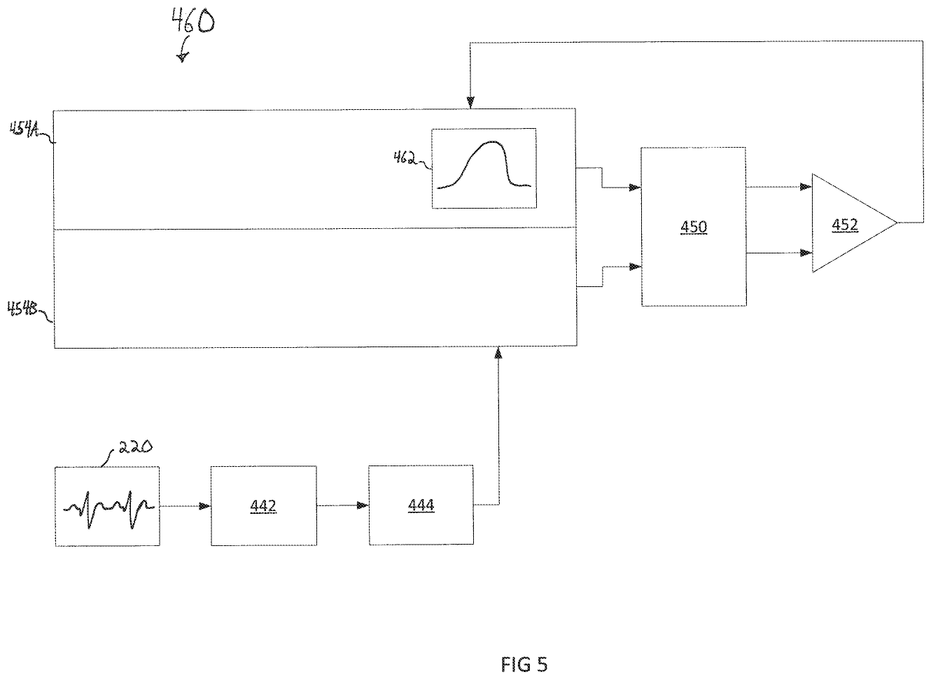

FIG. 5 illustrates the circuitry 460 of FIG. 4 after the higher quality P-wave has been determined. The P-wave filter 452, after having received the two P-waves and their associated quality scores as inputs, outputs the P-wave of higher quality (P-wave 462 in this case) to the memory segment 454a and overwrites the previous entry. In some cases, the P-wave 464 remains in the memory segment 454a if it had been of higher quality than the P-wave 462 in this example. The characteristics of the higher quality P-wave 462 (e.g., rise time, slope, area under the curve, and duration) are calculated and used along with the P-wave 462 to characterize atrial conduction and calculate a likelihood of AF.

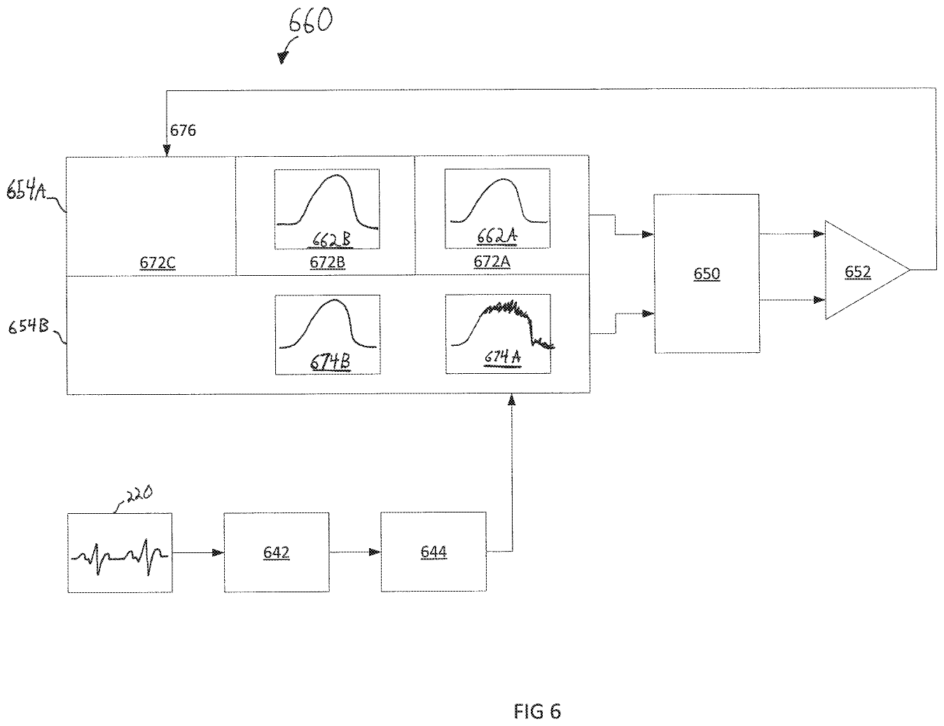

FIG. 6 illustrates, according to an illustrative implementation, circuitry 660 configured to compare the quality score of P-wave measurements. The circuitry 660 comprises an ECG signal interface 642, a P-wave extractor 644, a P-wave quality score calculator 650, a P-wave filter 652, and a memory pointer 676. The circuitry 660 also includes a memory segment 654a that is divided into a memory slot 672a that stores a P-wave 662a, a memory slot 672b that stores a P-wave 662b, and a memory slot 672c. The circuitry 660 also includes a memory segment 654b that stores a queue of extracted P-waves including a P-wave 674a and a P-wave 674b. The memory segment 654b stores a plurality of P-waves that have not yet been assessed for quality score and compared to other P-waves, such as the P-wave 674b, in a queue. The memory segment 654a contains multiple memory slots that store the high quality P-waves. The memory pointer 676 is directed to the last available memory slot 672c. In some implementations, the memory pointer 676 may be moved in accordance with performing operations on a data structure, such as a linked list, sorted list, dictionary, heap, or any other suitable structure, implemented within the memory 654a to store P-waves. In some implementations, the P-waves in memory segment 654a are ordered by P-wave quality score and the memory 654a stores a set or configurable number of high quality P-waves. In some implementations, the memory segment 654a stores a set or configurable number of P-waves that exceed a quality score threshold. In some implementations, the memory segment 354 associates the stored P-waves with a timestamp or timespan in which the ECG data was obtained from the patient. In some implementations, the memory segment 654a stores a high, or the highest, quality P-wave from a set of time periods, for example, by storing the highest quality P-wave extracted within each 30 second period, minute, 5 minutes, 30 minutes, hour, 6 hours, 24 hours, days, or weeks. The P-waves can reveal latent atrial defects that may later develop into AF or stroke, and fewer P-waves, or even only a single P-wave, may be required to detect such defects since the features of the P-wave are preserved. Requiring fewer P-waves can also yield faster diagnosis of AF and patient reports having a manageable number of waveforms.

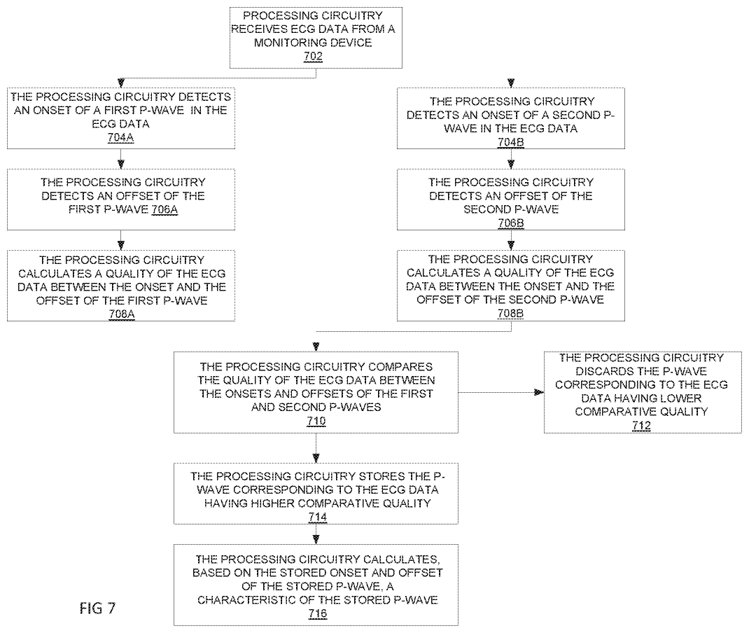

FIG. 7 shows, according to an illustrative implementation, a flow chart depicting a method 700 for obtaining P-wave signals and comparing their quality scores to detect atrial defects and predict atrial fibrillation. The method 700 is performed by processing circuitry, such as the processing circuitry 306 of FIG. 3, the monitor processing device 102 of FIG. 1, the processing system 106 of FIG. 1, or any other suitable processing system or device. In general, the processing circuitry obtains ECG data from a monitoring system (e.g., monitoring system 109 of FIG. 1), analyzes the ECG data, detects a first P-wave (e.g., P-wave 462 of FIG. 4) and a second P-wave (e.g., P-wave 464 of FIG. 4) within the ECG data, calculates a quality score for each P-wave, compares the quality score of each P-wave, and calculates the characteristics of the higher quality P-wave. As shown, the method includes receiving ECG data from a patient-portable monitoring device (step 702), detecting an onset of a first P-wave represented in the ECG data (step 704A), detecting an offset of the first P-wave (step 706A), calculating a quality score of the ECG data between the onset and the offset of the first P-wave (step 708A), detecting an onset of a second P-wave represented in the ECG data (step 704B), detecting an offset of the second P-wave (step 706B), calculating a quality score of the ECG data between the onset and the offset of the second P-wave (step 708B), comparing the quality score of the ECG data between the onset and offset of the first and second P-waves (step 710), discarding the P-wave corresponding to the ECG data having the lower quality score (step 12), storing the P-wave corresponding to the ECG data having the higher quality score (step 714), and calculating, based on the stored on set and offset of the stored P-wave, a characteristic of the stored P-wave (step 716).

At step 702, processing circuitry (e.g. processing circuitry 306) receives ECG data from a patient-portable monitoring device (e.g., monitoring device 101 worn by patient 110 or any suitable ECG monitoring device). The ECG data may be the result of continuous monitoring or may be periodically sampled for sufficient time to collect relevant data. At step 704A, the processing circuitry analyzes the ECG data and detects the onset of a first P-wave (e.g., P-wave 464). At step 706A, the processing circuitry analyzes the ECG data and detects the offset of the first P-wave. In the illustrative implementation, a P-wave extractor (e.g., P-wave extractor 344) uses a P-wave onset detector (e.g., P-wave onset detector 346) to determine the beginning of P-wave segments and a P-wave offset detector (e.g., P-wave offset detector 348) to determine the end of P-wave segments. In some implementations, the P-wave extractor uses a second biological signal, e.g. the Hilbert transform of the ECG signal, to form an analytic pair with the ECG signal. By analyzing the time varying phase angle of the analytic pair and defining reference lines, the P-wave extractor can identify the onset and offset of a P-wave. In some implementations, the P-wave onset detector defines an onset reference line and the P-wave offset detector defines an offset reference line.

At step 708A, the processing circuitry calculates the quality score of the ECG data between the onset and the offset of the first P-wave using a P-wave quality score calculator (e.g., P-wave quality score calculator 350) that calculates a quality score for the P-wave. In some implementations, the calculated quality score is an integer, real number, scalar value, percentage, noise measurement (e.g. an RMS noise power), or any other suitable measure that indicates the amount of noise in the P-wave. In some implementations, the processing circuitry rejects P-waves containing measurement defects or interference artifacts. The processing circuitry may compute the noise estimate and quality score for each P-wave by any suitable means. Next, the processing circuitry proceeds to step 704B.

At step 704B, the processing circuitry analyzes the ECG data and detects the onset of a second P-wave (e.g., P-wave 464). At step 706B, the processing circuitry analyzes the ECG data and detects the offset of the second P-wave. In the illustrative implementation, a P-wave extractor (e.g., P-wave extractor 344) uses a P-wave onset detector (e.g., P-wave onset detector 346) to determine the beginning of P-wave segments and a P-wave offset detector (e.g., P-wave offset detector 348) to determine the end of P-wave segments. In some implementations, the P-wave extractor uses a second biological signal, e.g. the Hilbert transform of the ECG signal, to form an analytic pair with the ECG signal. By analyzing the time varying phase angle of the analytic pair and defining reference lines, the P-wave extractor can identify the onset and offset of a P-wave. In some implementations, the P-wave onset detector defines an onset reference line and the P-wave offset detector defines an offset reference line.

At step 708B, the processing circuitry calculates a quality score of the ECG data between the onset and the offset of the second P-wave using a P-wave quality score calculator (e.g., P-wave quality score calculator 350) that calculates a quality score to the P-wave. In some implementations, the calculated quality score is an integer, real number, scalar value, percentage, noise measurement (e.g. an RMS noise power), or any other suitable measure that indicates the amount of noise in the P-wave. In some implementations, the processing circuitry rejects P-waves containing measurement defects or interference artifacts. The processing circuitry may compute the noise estimate and the quality score for each P-wave using any suitable means. In certain implementations, the processing circuitry performs steps 704A, 706A, and 708A in parallel with steps 704B, 706B, and 708B. Next, the processing circuitry proceeds to step 710.

At step 710, the processing circuitry compares the respective qualities of the ECG data between the onsets and offsets of the first and second P-waves. The processing circuitry processes the P-waves based on their associated quality scores using a P-wave filter (e.g. P-wave filter 352) that is configured to process P-wave signals based on their quality score. At step 712, the processing circuitry discards the P-wave corresponding to the ECG data having the lower quality score. As noted above, “discarding,” as used in this application, includes deleting, reallocating, overwriting, ignoring, removing, excluding, and/or taking no action on, data. The filter, which takes the first and second P-waves as input, may discard the lower quality P-wave. In some implementations, the processing circuitry compares each P-wave to a threshold quality score and discards the P-wave if the quality score does not exceed the threshold. In some implementations, the filter is configured as an ordered data structure that maintains a set or list of the highest quality P-waves identified by the processing circuitry. At step 714, the processing circuitry stores the P-wave corresponding to the ECG data having the higher quality score in memory (e.g., memory 354). In some implementations, the processing circuitry stores in memory one or more sufficiently high quality P-waves that may be held in a data structure ordered by quality score.

At step 716, based on the stored onset and offset of the stored P-wave, the processing circuitry calculates a characteristic of the stored P-wave. The processing circuitry may include a characteristic calculator (e.g., characteristic calculator 356), which calculates characteristics of the P-waves, such as the duration, the area under the curve, the rise time, a rate of rise, or any other suitable characteristic. The processing circuitry may calculate characteristics of the P-wave based on the P-wave as represented after the application of a waveform transform, a state-space transform, or any other suitable mathematical operation. The processing circuitry may combine, or average, high quality P-waves, or the characteristics thereof, in any suitable manner. The processing circuitry may communicate P-wave data to the AF risk calculator 358 and use the P-wave data to compute data relevant to AF. In some implementations, the data relevant to AF includes an atrial conduction velocity. In some implementations, the data relevant to AF includes a likelihood of AF. The AF risk calculator 358 may use a machine learning classifier, statistical regression, or any other suitable means to calculate AF likelihood and atrial conduction velocity. In some implementations, the calculated characteristics of the P-wave, AF likelihood, and atrial conduction velocity are graphically, or pictographically, presented. Although the processing circuitry may average P-waves, the systems and methods of the present disclosure allow for the use of un-averaged P-wave samples. For example by selecting a set of high quality P-waves, or a single high quality P-wave, averaging techniques may not be necessary. Using un-averaged P-wave data allows for a high resolution to be achieved because the high frequency information in the signal is not eliminated or distorted by the smoothing effects of averaging or other processing. The systems and methods of the present disclosure can also reduce or eliminate the effects of low quality samples distorting a composite P-wave.

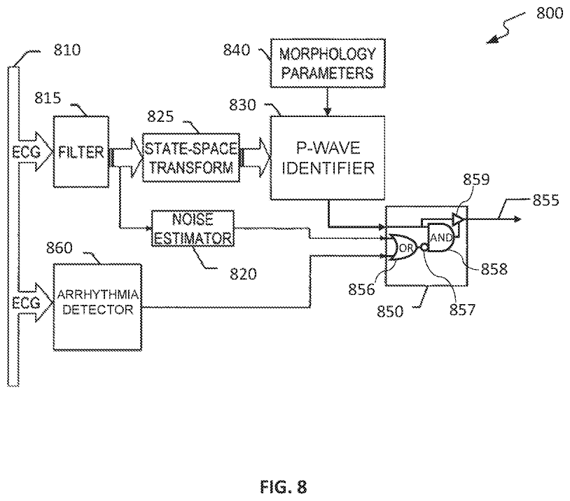

FIG. 8 shows, according to an illustrative implementation, a block diagram illustrating a P-wave detector 800 in a cardiac monitoring apparatus (e.g., processing circuitry 306 of FIG. 3, the monitor processing device 102 of FIG. 1, the processing system 106 of FIG. 1). The P-wave detector 800 includes an ECG input element 810, a filter 815, a noise estimator 820, a state-space transformation component 825, a P-wave identification component 830, a morphology parameters database, an arrhythmia detector 860, and final decision logic 850. The ECG input element 810 includes a split output that provides an ECG signal to two processing paths within the P-wave detector, the P-wave detection processing path and the arrhythmia detection processing path. The filter 815 filters the ECG signal received from the ECG input element 810 to clean the ECG signal as needed for later analytical processing. The filter 815 also further splits the ECG signal into separate signals for later parallel processing by the noise estimator 820 and the state-space transformation component 825. In some implementations, the filter 815 is a filter bank, which includes a baseline shift remover, one or more band pass filters, and/or any other suitable filter configured to prepare the ECG signal for later processing. The filter 815 may include an analog-to-digital converter.

The output of the filter 815 is provided to the noise estimator 820 and the state-space transformation component 825. The state-space transformation component 825 generates a partial state-space. The partial state space may be achieved by generating an analytical signal including an ECG signal and a transformation (e.g., Hilbert transformation) of the ECG signal. The output of the state-space transformation component 825 is provided to the P-wave identification component 830 for identification of P-waves.

The state-space transform, is based on the understanding of the heart as a dynamical system and governed by the deterministic (dynamical) laws governing the electrical pulses traveling through the heart tissue. For diagnostic purposes, a full reconstruction of the heart dynamics is often not necessary. Even partial reconstruction of the state-space, representing coarse-grained dynamics of the heart, can be a highly effective approach to cardiac monitoring. For example, in a full detailed state-space, the reconstructed dynamics may have a large amplitude when projected in some directions, and very small amplitude in other directions. By choosing a linear orthogonal transform that maximizes amplitude in two dimensions and minimizes the remaining projections, the first two dimensions represent large amplitude, coarse-grained dynamics, and other dimensions include lower amplitude, finer-grained dynamical movements. (An orthogonal transformation may be desirable because it preserve lengths of vectors and the angles between vectors.) In addition, because the finer-grained dimensions include noisy, less regular movements, the first two dimensions are typically the most useful for diagnostic purposes because they predominantly represent dynamics of the biological system (the heart, in this example) and are less influenced by noise. As should be appreciated in light of the above, instead of implementing a sequence of input signal→full state-space→two dimensional coarse-grained state-space, all that is needed is a direct transform of input signal→two dimensional coarse grained state-space, which should provide substantially all the benefits of the first in a less computationally intensive procedure.

To achieve partial state-space reconstruction, an analytical representation of the ECG signal is generated from an analytical pair, including an ECG signal and a transform of the ECG signal. The transformed signal is mathematically orthogonal (or effectively orthogonal) to the ECG signal, and the transform is frequency-independent in that it does not favor or amplify some frequencies of the signal over others. Generating the analytical representation from the underlying ECG signal is referred to as “embedding” the ECG signal into (partial) state-space. Any suitable frequency-independent transform, such as the Hilbert transform, can be used to embed the ECG signal in the analytical representation.

Frequency-independence is particularly useful in analyzing biological signals, such as ECG data, because such transforms preserve high frequency components of signals (e.g., frequencies of >5 Hz, >10 Hz, >20 Hz, >30 Hz, >40 Hz, >50 Hz, >60 Hz, >70 Hz, >80 Hz, >90 Hz, or >100 Hz). The heart's frequency spectrum can include frequencies as low as 1 Hertz and as high 100 Hertz. Moreover, frequency-independent transforms can be noise-insensitive compared to the original data in the time domain. This can be of great value when analyzing signals sensed from biological systems, where the noise component of the signal may be significant. Additionally, because the transform is noise-insensitive, in some implementations, the transform is applied directly to the unfiltered, or partially unfiltered, ECG signal from ECG input element 810. In other words, the transform can be applied at the front-end of the algorithm, rather than to some derivative of the cardiac signal. Furthermore, the state-space transformation performed by component 825 may achieve noise cancellation instead of, or in addition to, filter 815.

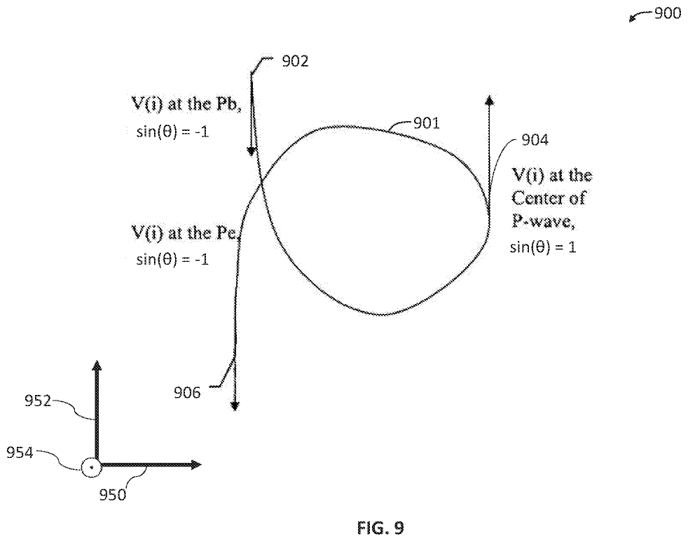

The P-wave identification component 830 receives the output of the state-space transformation component 825. The P-wave identification component 830 performs signal analysis in the partial state-space based on morphology parameters from the morphology parameter database 840. The P-wave identification component 830 identifies P-waves using the process described in relation to FIG. 9 below. In short, the P-wave identification component 830, uses a phase property of the partial state-space reconstruction along with empirically determined thresholds to determine the onset and offset of the P-wave. More specifically, the P-wave identification component 830 calculates the speed of trajectory, v(t), using finite differences as:

$$v ( t ) = \frac{z ( t + k ) - z ( t - k )}{2 k + 1}$$

where

z(t)=x(t)+i*H(x(t))

where t is discrete time, x(t) is the ECG signal, H(x(t)) is the Hilbert transform of the ECG signal, and z(t) is the function describing the analytical signal 901. The phase of v(t) can be computed as follows:

$$θ = {tan}^{- 1} ( \frac{I \{ v ( t ) \}}{R \{ v ( t ) \}} ) = {tan}^{- 1} ( \frac{H ( x ( t + k ) ) - H ( x ( t - k ) )}{x ( t + k ) - x ( t - k )} )$$

where I{v(t)} is the imaginary components of v(t), and R{v(t)} is the real component of v(t). The onset and offset of P-waves can be located based on a phase property of the speed of trajectory and two thresholds. As will be described further in relation to FIG. 9, the peak of the P-wave can be located as the closest maximum of sin(θ(i)) to the left of the Q-point of the QRS complex, which maximum is also above a certain threshold T_{P}. The threshold T_{P} is an empirically derived number, typically close to 1 (e.g., T_{P}=0.9). Consequently, the sine of the phase θ(i) descends to the left and to the right from the peak 904 of the analytical signal 901. The P-wave boundaries P_{b} at start point 902 and P_{c} at end point 906 can be located as the points at which sin(θ(i)) reaches a negative threshold value T_{Pb} (e.g., T_{Pb}=−0.9), or minimum (i.e., −1). The physical meaning of this approach is shown by the shape of the analytical signal 901 in FIG. 9 below.

In addition to using the phase of the speed of trajectory, as described above, the phase can also be used in functional combinations with other parameters, such as the amplitude of the partial state-space trajectory, |z(t)|. Since phase is not defined when z(t)=0, the discontinuities in the phase can be suppressed, for example, by the product

|z(t)|*sin(θ(t))

This represents a combination of a phase property of the speed of trajectory, v(i), and an amplitude property of the partial state-space trajectory, z(i). Moreover, this combination generally has extremes that point to wave form boundaries in the PQRST complex.

The noise estimator 820 estimates the level of noise in the filtered ECG signal received from the filter 815. The noise estimator 820 outputs a level of noise in the filtered ECG signal to the final decision logic 850. In some implementations, the noise estimator 820 outputs binary values: 1 if there is noise above a noise threshold and 0 if there is noise below the noise threshold. The arrhythmia detector 860 detects arrhythmia events present in the ECG signal received from the ECG input element 810. The arrhythmia detector 860 outputs an indication of arrhythmia events present in the ECG signal. In some implementations, the arrhythmia detector 860 outputs binary values: 1 if arrhythmia is detected and 0 if no arrhythmia is detected.

The final decision logic 850 receives the output of the P-wave identifier 830, the noise estimator 820, and the arrhythmia detector 860, and then outputs identified P-waves 855. The final decision logic includes an OR gate 856, an inverter 857, an AND gate 858, and a tristate buffer 859. The OR gate 856 receives outputs from the noise estimator 820, and the arrhythmia detector 860 and outputs a 1 if either of the inputs are 1. If both of the inputs to the OR gate 656 are zero, the OR gate 656 outputs a 0. The output of the OR gate 656 is inverted by the inverter 857 and then passed into the AND gate 858. The AND gate 858 also receives the output of the P-wave identifier 830. The AND gate 858 outputs a 1 if, and only if, the P-wave identifier detects a P-wave and the OR gate output is zero (since the output of the OR gate 856 is inverted before being passed to the AND gate 858). In other words, the output of the AND gate 858 is determined by the following logic:

[P-wave detected]∧¬([noise detected]∨[arrhythmia detected])

If the expression is true, then the AND gate 858 outputs a 1. Otherwise, the AND gate 858 outputs a 0. The output of the AND gate 858 is passed to the tristate buffer 859. If the output of the AND gate 858 is 1, then the tristate buffer 859 outputs the detected P-wave. If the output of the AND gate is 0, then the tristate buffer outputs 0. Thus, the final decision logic 850 only allows a detected P-wave to pass to the output 855 if the P-wave did not occur during a detected cardiac arrhythmia event or a detected noise event. The P-waves provided via the output 855 are thus of high quality and can used in downstream processing to determine P-wave characteristics such as a rate of rise of the P-wave, an area under the curve of the P-wave, a duration of the P-wave, and any other suitable measurement indicative of atrial structure, or any combination thereof. Although the P-wave identification component 830 is depicted as providing a single output to both the AND gate 858 and the tristate buffer 859, in practice it is preferable for the P-wave identification component 830 to provide a binary output (indicating 1 if, and only if, a P-wave is present) to AND gate 858 and a separate output (indicating the voltage of the P-wave in analog or non-binary digital format) to the tristate buffer 859. This preferred approach avoids discontinuities that would occur if a portion of a detected P-wave is at or near 0V. If an analog signal representing the P-wave is merely split and fed into the AND gate 858, any 0V portion of the P-wave will be treated erroneously as a logical zero.

In some implementations, the P-wave detector 800 is a real-time P-wave detector that identifies successive P-waves in real time (i.e., output data is generated directly from live input data). The various components of the P-wave detector 800 can be implemented as analog or digital components or as software. For example, in certain implementations, the final decision logic is implemented using software. In certain implementations, the P-wave detector 800 processes stored ECG data (e.g., ECG data stored digitally in a computer database).

FIG. 9 shows an example plot 900 of an analytical representation 901 of an ECG signal of a P-wave over time in a state-space. The plot 900 includes a P-wave peak 904, start time 902, and end time 906. The plot 900 includes an x-axis 950, a y-axis 952, and a z-axis 954. The x-axis 950 represents the amplitude of an ECG signal. The units of the x-axis are millivolts. The y-axis 952 represents an amplitude of a transformed ECG signal. The z-axis 954 represents time in seconds. The z-axis 954 is normal to the page and pointing up (out of the page). In this example, a positive (or bipolar) P-wave is described. However, as will be readily understood, the general techniques described herein can be readily modified to handle, and are equally applicable to, all kinds of P-wave morphologies.

As noted above, the heart can be considered as a dynamical system, meaning that there are some deterministic (dynamical) laws governing the electrical pulses traveling through the heart tissue. Partial reconstruction of the state-space, representing coarse-grained dynamics of the heart, can be a highly effective approach to cardiac monitoring. By choosing a linear orthogonal transform that maximizes amplitude in two dimensions and minimizes the remaining projections, the first two dimensions represent large amplitude, coarse-grained dynamics, and other dimensions include lower amplitude, finer-grained dynamical movements. Thus, the first two dimensions should be the most useful for diagnostic purposes because they predominantly represent dynamics of the heart and are less influenced by noise. Therefore, the first two dimensions can be used for diagnostic purposes.

The analytical representation 901 is such a partial state-space reconstruction generated from an analytical pair comprising an ECG signal and a transform of the ECG signal. The transformed signal is mathematically orthogonal (or effectively orthogonal) to the ECG signal, and the transform is frequency-independent in that it does not favor or amplify some frequencies of the signal over others. Generating the analytical representation 901 from the underlying ECG signal is referred to as “embedding” the ECG signal into (partial) state-space. The Hilbert transform, a type of frequency-independent transform, was used to embed the ECG signal in the analytical representation 901 of FIG. 9. The embedding can be achieved using any other suitable frequency-independent transform. Frequency-independence is particularly useful in analyzing biological signals, such as ECG data, because such transforms preserve high frequency components of signals (e.g., frequencies of >5 Hz, >10 Hz, >20 Hz, >30 Hz, >40 Hz, >50 Hz, >60 Hz, >70 Hz, >80 Hz, >90 Hz, or >100 Hz). The heart's frequency spectrum can include frequencies as low as 1 Hertz and as high 100 Hertz. Moreover, frequency-independent transforms can be noise-insensitive. This can be of great value when analyzing signals sensed from biological systems, where the noise component of the signal may be significant.

The analytical representation 901 is a two-dimensional partial state-space reconstruction (the original signal plus its Hilbert transform, with the third dimension of time being implicit). A partial state-space approach can be sufficient for signals in which a few major wave modes dominate the signal (e.g., typical ECG signals). The two-dimensionality of the partial state-space has the advantage of reducing the complexity of automated analysis compared to higher dimensional state-space reconstructions. Additionally, in some implementations, only a single input signal is needed to generate the analytical signal 901 since it is only two dimensional. Thus, although the Hilbert transform does not achieve a full-state-space reconstruction, the Hilbert transform can still be useful for ECG analysis.

The analytical signal 901 retains many properties of the original signal, while also adding properties specific to the state-space representation. For example, noise in the original signal tends to have increasingly different/irregular dynamical behavior in the analytical signal 901 compared to the ECG signal. Thus, detecting, estimating, and filtering noise can be easier using the analytical signal 901 compared to the underlying ECG signal.

The analytical signal 901 can be employed to extract physiological information for the heart which is not apparent from the underlying ECG signal directly. For example, the analytical signal can be used to calculate three or more dynamical measures of heart activity and derivative physiological quantities, such as speed of trajectory, length of trajectory, area integral of a speed vector and threshold crossings in state-space. The calculation of speed of trajectory is particularly helpful for detecting P-waves. The speed of trajectory, v(t), can be calculated using finite differences as:

$$v ( t ) = \frac{z ( t + k ) - z ( t - k )}{2 k + 1}$$

where

z(t)=x(t)+i*H(x(t))

where t is discrete time, x(t) is the ECG signal, H(x(t)) is the Hilbert transform of the ECG signal, and z(t) is the function describing the analytical signal 901. Note that v(t) is a complex number, having an amplitude, r, and phase, θ, on the complex plane. The phase of v(t) can be expressed as follows

$$θ = {tan}^{- 1} ( \frac{I \{ v ( t ) \}}{R \{ v ( t ) \}} ) = {tan}^{- 1} ( \frac{H ( x ( t + k ) ) - H ( x ( t - k ) )}{x ( t + k ) - x ( t - k )} )$$

where I{v(t)} is the imaginary components of v(t), and R{v(t)} is the real component of v(t). In general, the peak and boundaries of a P-wave can be located based on a phase property of the speed of trajectory and two thresholds. For example, in the analytical signal 901, the sine of the phase angle can be used to determine the start point 902, end point 906, and peak 904 of the P-wave. The peak of the P-wave can be located as the closest maximum of sin(θ(i)) to the left of the Q-point of the QRS complex, which maximum is also above a certain threshold T_{P}. The threshold T_{P} is an empirically derived number, typically close to 1 (e.g., T_{P}=0.9). Consequently, the sine of the phase θ(i) descends to the left and to the right from the peak 904 of the analytical signal 901. The P-wave boundaries P_{b} at start point 902 and P_{c} at end point 906 can be located as the points at which sin(θ(i)) reaches a negative threshold value T_{Pb} (e.g., T_{Pb}=−0.9), or minimum (i.e., −1). The physical meaning of this approach is shown by the shape of the analytical signal 901. As shown in FIG. 9, the analytical signal 901 performs a full phase rotation through the course of the P-wave. Although a finite difference method of calculation is discussed above, it should be apparent that the speed of trajectory is the derivative of the analytical signal and that the finite difference method merely estimates the derivative over multiple (2*k) samples. Other computation methods, including other finite difference methods, can be used instead to approximate v(t).

In practice, the degree of phase accumulation and the position of the analytical signal 901 at the peak 904 of a P-wave may vary depending on P-wave morphology and possible collision with the neighboring wave forms (like T-wave from the preceding heart beat). However, in a typical embodiment, the parameters T_{P} and T_{Pb} can be selected universally to accommodate for the majority of morphologies and ranges of heart rates.

In addition to using the phase of the speed of trajectory, as described above, the phase can also be used in functional combinations with other parameters, such as the amplitude of the partial state-space trajectory, |z(t)|. Since phase is not defined when z(t)=0, the discontinuities in the phase can be suppressed, for example, by the product:

|z(t)|*sin(θ(t))

This represents a combination of a phase property of the speed of trajectory, v(i), and an amplitude property of the partial state-space trajectory, z(i). Moreover, this combination generally has extremes that point to waveform boundaries in the PQRST complex. Thus, this combination (and variations of it) can be valuable in automated cardiac analysis since it readily identifies waveform boundaries by having its extremes at the points of waveform change.

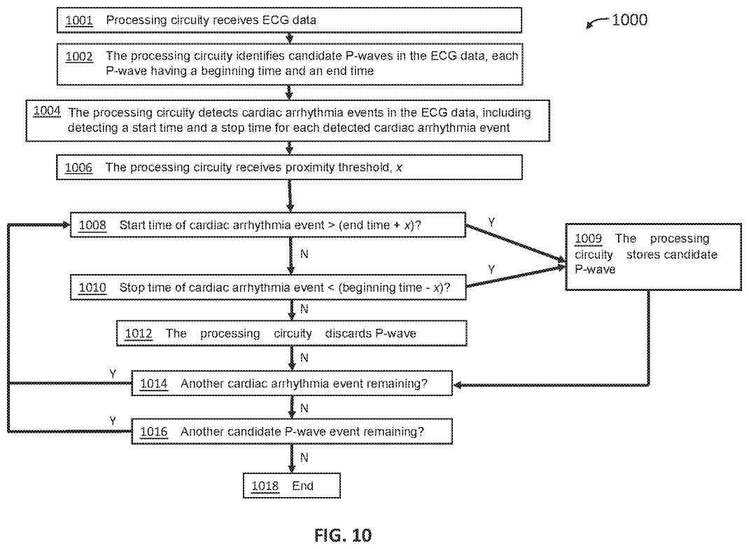

FIG. 10 shows, according to an illustrative implementation, a flow chart of a method 1000 for determining whether P-waves are concurrent with stored cardiac arrhythmia events. By determining whether a P-wave is concurrent with a cardiac arrhythmia event (e.g., AF, cardiac pause, ventricular fibrillation), the method can exclude from analysis P-waves that may not be representative of cardiac function.

In step 1001, the processing circuitry (e.g., processing circuitry 306 of FIG. 3, the monitor processing device 102 of FIG. 1, the processing system 106 of FIG. 1, or any other suitable processing system or device) receives ECG data. In step 1002, the processing circuitry detects candidate P-waves. The processing circuitry performs the P-wave detection using the partial state-space reconstruction approach described in relation to FIG. 9. Each P-wave has a beginning time and an end time which are determined using the phase angle of that partial state-space reconstruction. In step 1004, the processing circuitry detects cardiac events in the ECG data. Each detected cardiac event includes a start time and a stop time. The cardiac arrhythmia events can be detected using the arrhythmia detector 860 of FIG. 8 or any other suitable system. In step 1006, the processing circuitry receives a proximity threshold, x. The processing circuitry uses the proximity threshold to determine “near concurrence” and “strict concurrence” as opposed to only strict concurrence. As used herein, concurrence includes strict concurrence and near concurrence. Strict concurrence occurs when two events (e.g., a P-wave and an arrhythmia event) overlap at some point in time. Near concurrence occurs when two events do not overlap at any point in time, but occur very near in time to each other. In other words, near concurrence is present when two events, though not overlapping, are within x units of time of each other, where x is the proximity threshold, which is a small number. In some implementations, x is 10 minutes, 5 minutes, 2 minutes, 1 minute, 30 seconds, 15 seconds, 10 seconds, 5 seconds, 4 seconds, 3 seconds, 2 seconds, 1 second, 0.5 seconds, 0.1 seconds, 1 beat cycle, or any other suitable number. In some implementations, x=0 so that only strict concurrence will be detected.

In step 1008, for each detected cardiac event, the processing circuitry evaluates the following expression (Expression 1):

[start time of cardiac event]ᵢ>(end timeⱼ+x)

Here, i denotes the i^{th} cardiac arrhythmia event and j denotes the j^{th} P-wave. The Expression 1 may be computed for every combination of cardiac arrhythmia event and P-wave (e.g., computed i*j times). If Expression 1 is true, then the cardiac arrhythmia event i began so much later than the P-wave j that the cardiac arrhythmia event i is not concurrent with the P-wave j. Thus, if Expression 1 is true, the P-wave is stored at 1009, because the cardiac arrhythmia event i and the P-wave j are not concurrent. If Expression 1 is false, then there is a chance that the cardiac arrhythmia event i and the P-wave j are concurrent. To determine whether there is concurrence, it is necessary to examine the stop time of the cardiac event i using Expression 2:

[stop time of cardiac event]ᵢ<(beginning timeⱼ−x)

If Expression 2 is true, then the cardiac arrhythmia event i ended so much earlier than the P-wave j that the cardiac event i could not have been concurrent with the P-wave j. Thus, if Expression 2 is true, the P-wave is stored at step 1009 because the cardiac event i and the P-wave event j are not concurrent. If Expression 1 and Expression 2 are both false, then the cardiac arrhythmia event i and the P-wave j are concurrent (either strictly concurrent or nearly concurrent), and the method proceeds to discard the P-wave at step 1012 because the P-wave might be non-representative. The skilled person would appreciate that Expression 1 and Expression 2 can be evaluated in reverse order or even simultaneously. In any event, only if both Expression 1 and Expression 2 are false can the cardiac arrhythmia event i and the P-wave j be concurrent. If either Expression 1 or Expression 2 are true, then the cardiac arrhythmia event i and the P-wave j cannot be concurrent. In Expression 1 and Expression 2, later times are represented by higher values such that (end time+x) means x units of time after the end time. Conversely, (beginning time−x) means x units of time before the beginning time.

After the discarding in step 1012, or the storing in step 1009, the processing circuitry proceeds to step 1014. At step 1014, the processing circuitry determines whether another cardiac arrhythmia event remains to be evaluated against the P-wave j. If another cardiac arrhythmia event remains (e.g., cardiac arrhythmia event i+1), then the processing circuitry proceeds to step 1008 and repeats the concurrence analysis (e.g., steps 1008 and 1010). If no more cardiac arrhythmia events remain, the processing circuitry proceeds to step 1016. At step 1016, the processing circuitry determines whether any other P-waves remain. If one or more P-waves remain, the processing circuitry repeats the concurrence analysis for the next P-wave (e.g., P-wave j+1). If no additional P-waves remain, the processing circuitry proceeds to step 1018. At step 1018, the processing circuitry calculates the characteristics of the stored P-waves.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

1. An electrocardiogram (ECG) monitoring system comprising:
a pair of electrodes electrically coupled to an ECG sensor and configured to receive ECG data from a patient;
a computer-readable memory; and
wherein the ECG sensor is configured to:
receive the ECG data from the pair of electrodes;
detect an onset and an offset of a first candidate P-wave represented in the ECG data;
calculate a quality score of the first candidate P-wave by calculating a quality score of ECG data between the onset and the offset of the first candidate P-wave;
detect an onset and an offset of a second candidate P-wave represented in the ECG data;
calculate a quality score of the second candidate P-wave by calculating a quality score of ECG data between the onset and the offset of the second candidate P-wave;
compare the quality score of the first candidate P-wave with the quality score of the second candidate P-wave;
in response to determining, based on the comparison, that the first candidate P-wave has a lower quality score than the second candidate P-wave, discard the first candidate P-wave;
store, in the computer-readable memory, an indication of the second candidate P-wave;
calculate a characteristic of the second candidate P-wave, wherein the characteristic is at least one of a rate of rise of the stored P-wave, an area under the curve of the stored P-wave, and a duration of the stored P-wave.

2. The system of claim 1, wherein calculating the quality score of the first candidate P-wave comprises determining an average root mean square amplitude of noise between the onset and the offset of the first candidate P-wave.

3. The system of claim 1, wherein calculating the quality score of the first candidate P-wave comprises determining whether the first candidate P-wave coincides with an arrhythmia event.

4. The system of claim 2, wherein the circuitry is configured to detect an onset of the first candidate P-wave using a Hilbert transform.

5. The system of claim 4, wherein the circuitry is further configured to graphically present the calculated characteristic of the stored P-wave on a display.

6. The system of claim 5, wherein the circuitry is further configured to calculate an atrial conduction velocity based on the calculated characteristic of the stored P-wave.

7. The system of claim 6, wherein the circuitry is further configured to calculate an indication of a likelihood of atrial fibrillation or stroke based on the characteristic of the stored P-wave.

8. The system of claim 1, wherein the circuitry is further configure to store in the computer-readable memory a plurality of P-waves.

9. A system for filtering P-waves comprising:
processing circuitry configured to:
receive ECG data from an ECG monitoring device;
detect an onset and an offset of a candidate P-wave represented in the ECG data;
calculate a quality score of the candidate P-wave by calculating a quality score of ECG data between the onset and the offset of the candidate P-wave;
compare the quality score of the candidate P-wave with a quality score threshold;
if the quality score of the candidate P-wave is equal to or greater than the quality score threshold, store an indication of the candidate P-wave to computer-readable memory, and
calculate a characteristic of the stored P-wave, wherein the characteristic is at least one of a rate of rise of the stored P-wave, an area under the curve of the stored P-wave, and a duration of the stored P-wave.

10. The system of claim 9, wherein calculating the quality score of the candidate P-wave comprises determining whether the P-wave coincides with an arrhythmia event, and wherein the calculation of the quality score of the candidate P-wave is configured such that when the candidate P-wave does not coincide with an arrhythmia event, the calculated quality score of the candidate P-wave exceeds the quality score threshold.

11. The system of claim 9, wherein the quality score of the candidate P-wave is an indicator of whether the candidate P-wave includes an ECG artifact, and wherein the quality score of the candidate P-wave is equal to or greater than the quality score threshold when the P-wave does not include an ECG artifact.

12. The system of claim 9, wherein the quality score threshold is determined based on at least one of: an error in the rate of rise of the stored P-wave, the area under the curve of the stored P-wave, or the duration of the stored P-wave.

13. The system of claim 10, wherein the circuitry is further configured to graphically present the calculated characteristic of the stored P-wave in a display.

14. The system of claim 13, wherein the circuitry is further configured to calculate an indication of a likelihood of atrial fibrillation or stroke based on the characteristic of the stored P-wave.

15. The system of claim 9, wherein the circuitry is further configured to store a plurality of P-waves in computer-readable memory.

16. A method for filtering P-waves comprising:
receiving ECG data from an ECG monitoring device;
detecting an onset and an offset of a first candidate P-wave represented in the ECG data;
calculating a quality score of the first candidate P-wave by calculating a quality score of ECG data between the onset and the offset of the first candidate P-wave;
comparing the quality score of the first candidate P-wave with a quality score threshold;
if the quality score of the candidate P-wave is equal to or greater than the quality score threshold, storing the candidate P-wave;
if the quality score of the candidate P-wave is less than the quality score threshold, discarding the candidate P-wave; and
calculating a characteristic of the stored P-wave, wherein the characteristic is at least one of a rate of rise of the stored P-wave, an area under the curve of the stored P-wave, and a duration of the stored P-wave.

17. The method of claim 16, further comprising:
detecting an onset and an offset of a second candidate P-wave represented in the ECG data;
calculating a quality score of the second candidate P-wave by calculating a quality score of ECG data between the onset and the offset of the second candidate P-wave; and
setting the quality score threshold equal to the quality score of the second candidate P-wave.

18. The method claim 16, further comprising graphically presenting the calculated characteristic of the stored P-wave.

19. The method of claim 18, further comprising estimating an atrial conduction velocity based on the calculated characteristic of the stored P-wave.

20. The method of claim 16, further comprising estimating an indication of a likelihood of atrial fibrillation or stroke based on the characteristic of the stored P-wave.
